(19) United States
(12) Patent Application Publication
(10) Pub. No.: US 2020/0003773 A1
(43) Pub. Date: Jan. 2, 2020

(54) OPTICAL DETECTION OF BIOLOGICAL COMPONENTS

(71) Applicant: University of Massachusetts, Boston, MA (US)

(72) Inventors: Lili He, Belchertown, MA (US); Brooke Pearson, New York, NY (US)

(21) Appl. No.: 16/375,310

(22) Filed: Apr. 4, 2019

Related U.S. Application Data
(60) Provisional application No. 62/653,322, filed on Apr. 5, 2018.

(51) Int. Cl. G01N 33/553; G01N 33/569; G01N 21/65

(52) U.S. Cl. CPC G01N 33/553; G01N 21/658; G01N 33/56916; G01N 33/56911

(57) ABSTRACT

The present disclosure provides a substrate. The substrate includes a metallic layer. The substrate further includes a boronic acid component, a nucleic acid component, or both at least partially coating the metallic layer.

### CROSS-REFERENCE TO RELATED APPLICATION

This application claims the benefit of priority to U.S. Provisional Patent Application Ser. No. 62/653,322 entitled “OPTICAL DETECTION OF BIOLOGICAL COMPONENTS,” filed Apr. 5, 2018, the disclosure of which is incorporated herein in its entirety by reference.

### STATEMENT OF GOVERNMENT SUPPORT

This invention was made with Government support under 2015-67021-22993 awarded by the United States Department of Agriculture, National Institute of Food and Agriculture (USDA/NIFA). This invention was also made with Government support under 2016-31100-06025 awarded by the United States Department of Agriculture, National Institute of Food and Agriculture (USDA/NIFA) Hatch Capacity Funding. This invention was also made with Government support under NI17HFPXXXXXG052 awarded by the United States Department of Agriculture, National Institute of Food and Agriculture (USDA/NIFA) Hatch Capacity Funding. The U.S. Government has certain rights in this invention.

### BACKGROUND

The presence of bacteria in a food source or in an environment can be hazardous to health. To help reduce the risk of ingesting or being exposed to bacteria it is desirable to screen a food source or sample from the environment to determine whether bacteria is present. However, screening for bacteria can be complicated and require intricate systems that are difficult to operate and lack mobility to perform rapid on-site testing. It is desirable therefore, to develop systems and methods for rapid and easy optical detection of bacteria.

### SUMMARY OF THE DISCLOSURE

The present disclosure provides a substrate. The substrate includes a metallic layer. The substrate further includes a boronic acid component at least partially coating the metallic layer.

The present disclosure further provides a method of making a substrate. The substrate includes a metallic layer. The substrate further includes a boronic acid component at least partially coating the metallic layer. The method includes coating the metallic layer with the boronic acid component.

The present disclosure further provides a system for detection of a biological component. The system includes a substrate. The substrate includes a metallic layer. The substrate further includes a boronic acid component at least partially coating the metallic layer. The system further includes a magnifying lens in optical communication with the substrate.

The present disclosure further includes a method of using a substrate. The substrate includes a metallic layer. The substrate further includes a boronic acid component at least partially coating the metallic layer. The method includes contacting the substrate with an analyte. The method further includes exposing the substrate to light. The method further includes capturing an image of the substrate. The method further includes visually confirming the presence or absence of bacteria.

The present disclosure provides a substrate. The substrate includes a metallic layer. The substrate further includes a nucleic acid component at least partially coating the metallic layer.

The present disclosure further provides a method of making a substrate. The substrate includes a metallic layer. The substrate further includes a nucleic acid component at least partially coating the metallic layer. The method includes coating the metallic layer with the nucleic acid component.

The present disclosure further provides a system for detection of a biological component. The system includes a substrate. The substrate includes a metallic layer. The substrate further includes a nucleic acid component at least partially coating the metallic layer. The system further includes a magnifying lens in optical communication with the substrate.

The present disclosure further includes a method of using a substrate. The substrate includes a metallic layer. The substrate further includes a nucleic acid component at least partially coating the metallic layer. The method includes contacting the substrate with an analyte. The method further includes exposing the substrate to light. The method further includes capturing an image of the substrate. The method further includes visually confirming the presence or absence of bacteria.

There are several advantages associated with the substrates, systems, and methods of the present disclosure, some of which are unexpected. According to some embodiments, the boronic acid component or the nucleic acid component of the substrate provides an attractive and at least somewhat selective binding target for bacteria. Nucleic acids can be particularly well suited for selective binding to target bacteria. According to some embodiments, the presence of bacteria bound to the boronic acid component or nucleic acid of the substrate can be visually confirmed under relatively low magnification power. According to some embodiments, the bacteria can be imaged conveniently with a relatively low magnification power lens attached to a camera of a smartphone, tablet, or computer. According to some embodiments, the image can be conveniently processed using a program or application associated with the smartphone, tablet, or computer. According to some embodiments, the presence or absence of bacteria on a food sample such as meat or a vegetable can be visually confirmed by contacting the food sample, or a portion thereof, with the substrate and imaging the substrate. According to some embodiments, the ability to conveniently confirm the presence or absence of bacteria in a food sample can allow for onsite screening of food samples for safety prior to consumption. According to some embodiments, the presence or absence of bacteria in an environmental sample, such as a pond or a soil sample, can be visually confirmed by contacting the environmental sample, or a portion thereof, with the substrate and imaging the substrate. According to some embodiments, the ability to conveniently confirm the presence or absence of bacteria can be applied to any aqueous sample. According to some embodiments, the systems and methods described herein can be incorporated into a portable system than can be used for educational purposes or for onsite screening. According to some embodiments, where the boronic acid component of the substrate comprises 3-mercaptophenylboronic acid the presence or absence of bacteria is readily confirmed using a lower magnification than a corresponding substrate that is free of 3-mercaptophenylboronic acid. According to some embodiments, images of the substrate can be pixilated and used to determine the concentration of the bacteria in the sample. According to some embodiments, the substrates can be used in conjunction with a surface enhanced Raman spectroscopy method to confirm the presence of the bacteria.

### BRIEF DESCRIPTION OF THE FIGURES

The drawings illustrate generally, by way of example, but not by way of limitation, various embodiments discussed in the present document.

FIG. 1 is a schematic diagram showing a portion of a substrate having a boronic acid component functionalized thereto interacting with a biological component as well as a visual display of the substrate at low magnification, in accordance with various embodiments.

FIG. 2A shows a photograph of a gold coated substrate, in accordance with various embodiments.

FIG. 2B shows a 3-mecapto boronic acid (3-MPBA)-coated substrate, in accordance with various embodiments.

FIG. 2C shows Salmonella enterica (SE 1045) 10⁸ CFU/mL in water dropped and dried on the substrate, in accordance with various embodiments.

FIG. 2D shows SE 1045 in 50 mM ammonia bicarbonate (pH 8.4) captured on 3-MPBA-coated substrate under the 20× objective lens, in accordance with various embodiments.

FIG. 2E shows an enlarged image of FIG. 2D, in accordance with various embodiments.

FIG. 2F, shows another enlarged image of FIG. 2D, in accordance with various embodiments.

FIG. 3A shows SE 1045 suspended in water, in accordance with various embodiments.

FIG. 3B shows SE 1045 suspended in sodium hydroxide (pH 8.4), in accordance with various embodiments.

FIG. 3C shows SE 1045 suspended in 50 mM ammonia bicarbonate (pH 8.4), in accordance with various embodiments.

FIG. 4A shows optical video images of a 100 μm×100 μm area of Salmonella enterica on a 3-MPBA sandwich substrate at a wide range of concentrations from 0 to 3.5×10⁷ CFU mL⁻¹), in accordance with various embodiments.

FIG. 4B shows optical analysis of adjacent video images, in accordance with various embodiments.

FIG. 4C shows the relationship between Salmonella enterica concentration and percent of optical image positive for ‘black dots’, in accordance with various embodiments.

FIG. 5 is a graph showing a comparison of bacteria counts within pond water between three different methods, SERS, optical, and culture quantification, in accordance with various embodiments.

FIG. 6A is a photograph of a negative control showing the presence of bacteria in chicken extract, in accordance with various embodiments.

FIG. 6B is a photograph showing the presence of bacteria in chicken extract, in accordance with various embodiments.

FIG. 7A shows a smartphone and microscopic adaptor that takes photos of bacteria captured on the substrate, in accordance with various embodiments.

FIG. 7B is a photograph taken by a smartphone and microscopic adaptor of a 3-MPBA-modified substrate control, in accordance with various embodiments.

FIG. 7C is a photograph taken by a smartphone and microscopic adaptor of a 3-MPBA-modified substrate with 10⁷ CFU/mL Salmonella cells, in accordance with various embodiments.

FIG. 8 is a schematic diagram showing a method a of making and using a substrate having nucleic acid aptamers functionalized thereto to selectively bind bacteria, in accordance with various embodiments.

FIG. 9A is a picture of an output showing bacteria functionalized to the aptamers of FIG. 8, in accordance with various embodiments.

FIG. 9B is a transformed image of the picture of FIG. 9A where bacteria are represented as pixels, in accordance with various embodiments.

FIG. 10. is a bar graph showing the amounts of pixels corresponding to bacteria captured using a substrate having a nucleic acid aptamer, in accordance with various embodiments.

FIG. 11. is another bar graph showing the amounts of pixels corresponding to bacteria captured using a substrate having a nucleic acid aptamer, in accordance with various embodiments.

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or trisubstituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “aryl” as used herein refers to cyclic aromatic hydrocarbon groups that do not contain heteroatoms in the ring. Thus, aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

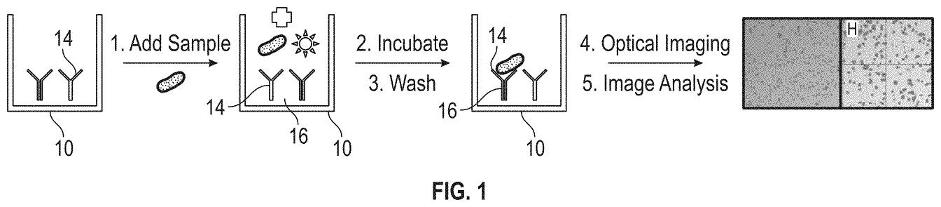

Various embodiments of the present disclosure relate to a substrate and system for optical detection of a biological component such as bacteria. The substrate can include many suitable components such as a metallic layer and a linking molecule functionalized to or otherwise bonded to the metallic layer and adapted to bind to the biological component. According to various embodiments, the linking molecule can be a boronic acid component or a nucleic acid aptamer. FIG. 1 is a schematic diagram showing a portion of substrate or chip 12 having boronic acid component 14 bonded thereto interacting with biological component 16 as well as a visual display of the substrate at low magnification. In an alternative embodiment, the substrate or chip can have a nucleic acid aptamer bonded thereto interacting with the biological component.

The substrate can include a metallic layer. In other embodiments, the metallic layer can be substituted with any other substantially non-transparent material such as a polymeric material. The metallic layer includes a lustrous or semi-conductive metal. For example, the metallic layer can include Ag₂O, elemental silver, elemental gold, elemental copper, elemental platinum, mixtures thereof, alloys thereof, or combinations thereof. The metallic layer can be a continuous layer, monolithic layer, or both. Alternatively, the metallic layer can be a discontinues layer that is applied to a backing layer. In embodiments in which the metallic layer is discontinuous, the metallic layer can include a collection of microparticles, nanoparticles, or mixtures thereof. Microparticles are generally understood to refer to particles individually having at least one dimeson (e.g., length, width, thickness, diameter, or height) in the micrometer range. The micrometer range can include any distance from about 1 μm to about 1000 μm, about 100 μm to about 500 μm, or less than, equal to, or greater than about 1 μm, 100, 200, 300, 400, 500, 600, 700, 800, 900, or about 1000 μm. Nanoparticles are generally understood to refer to individual particles having at least one dimension (e.g., length, width, thickness, diameter, or height) in the nanometer range. The nanometer range can include any distance from about 1 nm to about 10000 nm, about 100 nm to about 500 nm, or less than, equal to, or greater than about 1 nm, 1000, 2000, 3000, 4000, 5000, 6000, 7000, 8000, 9000, or about 10000 μm. Individual nanoparticles or microparticles can have any suitable morphology. For example, individual nanoparticles or microparticles may have a substantially spherical shape, a tubular shape, or a square shape.

In embodiments in which the metallic layer is a continuous layer, the metallic layer can have a substantially uniform thickness. Alternatively, the thickness of the metallic layer can be variable. The thickness of the metallic layer can be any suitable value. For example, at any point along metallic layer, the thickness can be in a range of from about 0.01 mm to about 5 mm, about 0.5 mm to about 2 mm, or less than, equal to, or greater than about 0.01 mm, 0.5, 1, 1.5, 2, 2.5, 3, 3.5, 4, 4.5, or about 5 mm. In the aggregate, an external surface of the metallic layer is substantially smooth. The smooth surface layer in combination with the lustrous nature of the metal can give the metal layer a high degree of reflectivity. For example, the metallic layer can reflect about 50% to about 99% of light to which it is exposed, about 80% to about 99%, or less than, equal to, or greater than about 50%, 55, 60, 65, 70, 75, 80, 85, 90, 95, or about 99%.

In some embodiments, the substrate can further include a coating or polymeric layer attached to the metallic layer. The coating or polymeric layer can include 6-mercapto-1-hexanol, polyvinylidene fluoride, polyethylene, polypropylene, polyvinyl chloride, polystyrene, polyamide, polytetrfluoroethylene, thermoplastic polyurethane, copolymers thereof, or mixtures thereof. Including the polymeric layer can help to increase the flexibility of the substrate or serve as a suitable base that can help to decrease the amount of metal required in the substrate. This can help to decrease costs associated with production of the substrate. The coating or polymerica layer can further include a linear polymer having at least one thiol end group. Example of suitable linear polymers can include a octanethiol a decanethiol, or the like. According to some embodiments, the coating layer may be substantially transparent such that light can be transmitted through the coating layer.

According to some embodiments, the coating layer can be used as a backfill to help the linking molecule stand. This can be particularly helpful when the linking molecule is a nucleic acid, which can be a large molecule. According to various embodiments, the coating layer can fill around the base of the aptamer to help stabilize the aptamer and help it to not lay flat on the metallic layer.

Furthermore, according to various embodiments adding the coating layer can be helpful to substantially block the metallic layer such that undesired molecules cannot functionalize to the metallic layer. As discussed further herein, some linking molecules, such as the nucleic acid aptamers selectively bind to certain bacteria as opposed to others. Therefore, it can be desirable to prevent molecules that may bind to other bacteria to be able to functionalize to the metallic layer and cause false positive test results.

Where present, the boronic acid component at least partially coats the metallic layer. The boronic acid component can be bonded directly to the metallic layer or bonded to linker molecule that is bonded to the metallic layer. Once the boronic acid is coated to the metallic layer, the metallic layer can be considered to be functionalized. As described further herein, the boronic acid component helps to enhance the ability to optically detect the bacteria. It is possible that the substrate can include further chemicals that may provide similar or identical enhancement of the ability to optically detect bacteria.

The boronic acid component can include an alkyl boronic acid, an alkenyl boronic acid, a heteroaryl boronic acid, a salt thereof, or a mixture thereof. In some embodiments, the aryl boronic acid is an unsubstituted aryl boronic acid, a monosubstituted aryl boronic acid, a disubstituted aryl boronic acid, a trisubstituted aryl boronic acid, a tetrasubstitued aryl boronic acid, a pentasubstituted aryl boronic acid, a salt thereof, or a mixture thereof. In other embodiments, the boronic acid component is 2-bromophenylboronic acid, 3-bromophenylboronic acid, 4-bromophenylboronic acid, 2-chlorophenylboronic acid, 3-chlorophenylboronic acid, 4-chlorophenylboronic acid, 2-fluorophenylboronic acid, 3-fluorophenylboronic acid, 4-fluorophenylboronic acid, 2-iodophenylboronic acid, 3-iodophenylboronic acid, 4-iodophenylboronic acid, 2-nitrophenylboronic acid, 3-nitrophenylboronic acid, 4-nitrophenylboronic acid, 3-mercaptophenylboronic acid, 4-mercaptophenylboronic acid, 2-hydroxyphenylboronic acid, 3-hydroxyphenylboronic acid, 4-hydroxyphenylboronic acid, 3-aminophenylboronic acid, (4-aminosulfonylphenyl)boronic acid, 3-boronobenzenesulfonamide, benzene-1,4-diboronic acid, (4-chlorocarbonylphenyl)boronic anhydride, 2,2-difluoro-benzo[1,3]dioxole-5-boronic acid, 2-(trifluoromethyl)phenylboronic acid, 3-(trifluoromethyl)phenylboronic acid, 4-(trifluoromethyl)phenylboronic acid, 2-(trifluoromethoxy)phenylboronic acid, 3-(trifluoromethoxy)phenylboronic acid, 4-(trifluoromethoxy)phenylboronic acid, 2-cyanophenylboronic acid, 3-cyanophenylboronic acid, 4-cyanophenylboronic acid, 2-formylphenylboronic acid, 3-formylphenylboronic acid, 4-formylphenylboronic acid, 2-(bromomethyl)phenylboronic acid, 3-(bromomethyl)phenylboronic acid, 4-(bromomethyl)phenylboronic acid, 3-boronobenzothioamide, 2-aminocarbonylphenylboronic acid, 3-aminocarbonylphenylboronic acid, 4-aminocarbonylphenylboronic acid, 2-(methylthio)phenylboronic acid, 3-(methylthio)phenylboronic acid, 4-(methylthio)phenylboronic acid, 2-(methylsulfonyl)phenylboronic acid, 3-(methylsulfonyl)phenylboronic acid, 4-(methylsulfonyl)phenylboronic acid, N-4-methanesulfonamidephenylboronic acid, 4-(cyanomethyl)benzeneboronic acid, 2-methoxycarbonylphenylboronic acid, 3-methoxycarbonylphenylboronic acid, 3-methoxycarbonylphenylboronic acid, 2-acetamidophenylboronic acid, 3-acetamidophenylboronic acid, 4-acetamidophenylboronic acid, 3-ethylsulfinylphenylboronic acid, 4-ethylsulfinylphenylboronic acid, or mixtures thereof. In some embodiments the boronic acid component can be free of 4-mercaptophenylboronic acid.

In some embodiments, the structure of the boronic acid is a structure according to any of Formula (I), Formula (II), a salt thereof, or mixtures thereof:

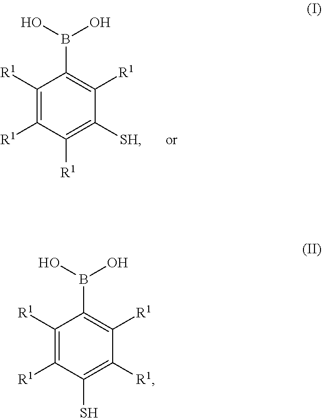

At each occurrence, R¹ is independently chosen from —H, —OH, —NH₂, substituted or unsubstituted (C₁-C₂₀)hydrocarbyl, and combinations thereof. In some embodiments the substituted or unsubstituted (C₁-C₂₀)hydrocarbyl is chosen from (C₁-C₂₀)alkyl, (C₁-C₂₀)alkenyl, (C₁-C₂₀)alkynyl, (C₁-C₂₀)acyl, (C₁-C₂₀)cycloalkyl, (C₁-C₂₀)aryl, and (C₁-C₂₀)alkoxy, and combinations thereof. In some embodiments at each occurrence R¹ is —H.

Where present as the capturer, nucleic acid aptamers are selectively engineered single-stranded deoxyribonucleic acid (DNA) or ribonucleic acid (RNA) that can bind to a specific target bacteria. They are relatively easy to create and manipulate, and often offer more stability, as well as improved sensitivity and robustness compared to antibodies. There are several published aptamer sequences for specific food pathogens. (See, for example, McKeague, M., Giamberardino, A., and DeRosa, M. C. (2011) Advances in aptamer-based biosensor for food safety, in Environmental biosensors (Somerset, V., Ed.), pp 17-42, which is incorporated by reference incorporated by reference herein in its entirety and for all purposes.). According to various embodiments, an example of a suitable single stranded DNA aptamer includes any of the sequences below, identified as SEQ ID 1 and SEQ ID 2:

SEQ ID 1
5′-S-S-AAA AAA CTC CTC TGA CTG TAA CCA CGC ACA AAG
GCT CGC GCA TGG TGT GTA CGT TCT TAC AGA GGT-3′
SEQ ID 2
5′-S-S-TTT TTT TTT TAT CCATGG GGC GGA GAT GAG GGG
GAG GAG GGC GGG TAC CCG GTT GAT-3

In some embodiments, the substrate can be at least partially disposed in a liquid solution. The liquid solution can include ethanol. Alternatively, the liquid solution can be an aqueous solution. The aqueous solution can include a base or a salt. For example, the aqueous solution can include a bicarbonate component. The bicarbonate component can include sodium bicarbonate, potassium bicarbonate, cesium bicarbonate, magnesium bicarbonate, calcium bicarbonate, ammonium bicarbonate, or mixtures thereof. Where present, the bicarbonate component can be in a range of from about 1 mM to about 100 mM, about 40 mM to about 60 mM, or less than, equal to, or greater than about 1 mM, 5, 10, 15, 20, 25, 30, 35, 40, 45, 50, 55, 60, 65, 70, 75, 80, 85, 90, 95, or about 100 mM. The amount of bicarbonate in the aqueous solution can bring the pH of the aqueous solution into the basic range. For example, the pH of the aqueous solution can be in a range of from about 7 to about 12, about, 8 to about 10, or less than, equal to, or greater than about 7, 7.5, 8, 8.5, 9, 9.5, 10, 10.5, 11, 11.5, or about 12. According to various further embodiments, the liquid solution can further include a phosphate-buffered saline (PBS) buffer. Where present the PBS can be diluted in water at a ratio in a range of from about 1.5:1 to about 1:1.5, about 1.2:1 to about 1:1.2, less than, equal to, or greater than about 1.5:1, 1.4:1, 1.3:1, 1.2:1, 1.1:1, 1:1, 1:1.1, 1:1.2, 1:1.3, 1:1.4, or about 1:1.5. According to various embodiments, where nucleic acids are used as the linking molecules, a liquid solution including the PBS can help to enhance the ability to detect bacteria.

The substrate can be used for detection of a biological component. The biological component can be included in the liquid solution. In some embodiments, the biological component includes one or more bacteria. Where present, the bacteria can be a gram-positive bacteria, a gram-negative bacteria, or a mixture thereof. In some embodiments, the bacteria can include Clostridium botulinum, Listeria monocytogenes, Acetic acid bacteria, Acidaminococcus, Acinetobacter baumannii, Agrobacterium tumefaciens, Akkermansia muciniphila, Anaerobiospirillum, Anaerolinea thermolimosa, Anaerolinea thermophila, Arcobacter, Arcobacter skirrowii, Armatimonas rosea, Azotobacter salinestris, Bacteroides, Bacteroides fragilis, Bacteroides ureolyticus, Bacteroidetes, Bartonella japonica, Bartonella koehlerae, Bartonella taylorii, Bdellovibrio, Brachyspira, Bradyrhizobium japonicum, Caldilinea aerophile, Cardiobacterium hominis, Chaperone-Usher fimbriae, Christensenella, Chthonomonas calidirosea, Coxiella burnetiid, Cyanobacteria, Cytophaga, Dehalogenimonas lykanthroporepellens, Desulfurobacterium atlanticum, Devosia pacifica, Devosia psychrophila, Devosia soli, Devosia subaequoris, Devosia submarina, Devosia yakushimensis, Dialister, Dictyoglomus thermophilum, Enterobacter, Enterobacter cloacae, Enterobacter cowanii, Enterobacteriaceae, Enterobacteriales, Escherichia, Escherichia coli, Escherichia fergusonii, Escherichia hermannii, Fimbriimonas ginsengisoli, Flavobacterium, Flavobacterium akiainvivens, Francisella novicida, Fusobacterium necrophorum, Fusobacterium nucleatum, Fusobacterium polymorphum, Haemophilus felis, Haemophilus haemolyticus, Haemophilus influenzae, Haemophilus pittmaniae, Helicobacter, Kingella kingae, Klebsiella pneumoniae, Kluyvera ascorbate, Kluyvera cryocrescens, Legionella, Legionella clemsonensis, Legionella pneumophila, Leptonema illini, Leptotrichia buccalis, Levilinea saccharolytica, Luteimonas aquatic, Luteimonas composti, Luteimonas lutimaris, Luteimonas marina, Luteimonas mephitis, Luteimonas vadose, Megamonas, Megasphaera, Meiothermus, Meiothermus timidus, Methylobacterium fijisawaense, Morax-Axenfeld diplobacilli, Moraxella, Moraxella bovis, Moraxella osloensis, Morganella morganii, Mycoplasma spumans, Neisseria cinereal, Neisseria gonorrhoeae, Neisseria meningitidis, Neisseria polysaccharea, Neisseria sicca, Nitrosomonas eutropha, Nitrosomonas halophila, Nonpathogenic organisms, OMPdb, Pectinatus, Pedobacter heparinus, Pelosinus, Propionispora, Proteobacteria, Proteus mirabilis, Proteus penneri, Pseudomonas, Pseudomonas aeruginosa, Pseudomonas luteola, Pseudoxanthomonas broegbernensis, Pseudoxanthomonas japonensis, Rickettsia rickettsia, Salinibacter ruber, Salmonella, Salmonella bongori, Salmonella enterica, Samsonia, Selenomonadales, Serratia marcescens, Shigella, Shimwellia, Solobacterium moorei, Sorangium cellulosum, Sphaerotilus natans, Sphingomonas gei, Spirochaeta, Spirochaetaceae, Sporomusa, Stenotrophomonas, Stenotrophomonas nitritireducens, Thermotoga neapolitana, Thorselliaceae, Trimeric autotransporter adhesion, Vampirococcus, Verminephrobacter, Vibrio adaptatus, Vibrio azasii, Vibrio campbellii, Vibrio cholerae, Victivallis vadensis, Vitreoscilla, Wolbachia, Yersiniaceae, Zymophilus, strains thereof, or combinations thereof. The bacteria can be a component of a sample from a food source. The food source can be a plant tissue or animal tissue. Alternatively, the bacteria can be a component of an environmental sample such as a sample of water.

The substrate can be at least partially disposed within a chamber such as a well. The well can be one well in a multi-well plate such as a 96-well plate. Alternatively, the chamber can include a wall or a plurality of walls bounding the substrate or a portion of the substrate. The chamber can help to contain the liquid solution within a specific region. Additionally, the presence of multiple chambers can be helpful to partition the substrate into different regions with different samples in each region. This can allow different samples to be tested for the presence of bacteria simultaneously.

Alternatively, the substrate can be located in a vial. Powdered buffer chemicals (e.g. the bicarbonate component, PBS, or NaOH, or a combination thereof) can be stored in a cap of the vial. The biological sample, which can be in an aqueous medium can then be contacted with the substrate. The buffer chemicals can be released from the cap and the vial can be shaken and left to incubate for any suitable amount of time. The substrate can then be removed, dried, and analyzed. In this manner the substrate can be part of a kit for the optical detection of bacteria.

The substrate can be manufactured according to any suitable method. For example, the substrate can be formed by coating the metallic layer with the boronic acid component or nucleic acid aptamer. Upon coating, a bond is formed between the metallic layer and the boronic acid component or nucleic acid aptamer. The metallic layer can be cleaned before coating. For example, the metallic layer can be cleaned with water or an organic solvent.

The boronic acid component or nucleic acid aptamer that is coated or functionalized on the metallic layer can be disposed in an aqueous solution. The concentration of the boronic acid or nucleic acid can be in a range from about 0.5 mM to about 5 mM, about, 0.5 mM to about 1.5 mM, or less than, equal to, or greater than about 0.5 mM, 1, 1.5, 2, 2.5, 3, 3.5, 4, 4.5, or about 5 mM. Coating or functionalizing can occur for over an amount of time ranging from about 1 hour to about 48 hours, about 10 hours to about 20 hours, or less than, equal to, or greater than about 1 hour, 5, 10, 15, 20, 25, 30, 35, 40, 45, or about 48 hours. After the metallic layer is coated or functionalized with the boronic acid component or nucleic acid aptamer, the substrate can be contacted with the bicarbonate component or PBS and the pH can be adjusted to an optimal value as described herein.

The substrate can be a component of a system for detection of a biological component such as bacteria. In addition to the substrate, the system can include a microscope system or magnification lens in optical communication with the substrate. The system can further include a light source. The magnification lens can magnify the substrate to any suitable degree. For example, the magnifying lens has a magnification range of from about 10× to about 300×, about 15× to about 250× about 15× to about 30×, less than, equal to, or greater than about 10×, 15×, 20×, 25×, 30×, 35×, 40×, 45×, 50×, 55×, 60×, 65×, 70×, 75×, 80×, 85×, 90×, 95×, 100×, 110×, 120×, 130×, 140×, 150×, 160×, 170×, 180×, 190×, 200×, 210×, 220×, 230×, 240×, 250×, 260×, 270×, 280×, 290×, or about 300×. Notably, the construction of the substrate can allow for adequate magnification towards the lower end of this range. Thus, the ability to optically detect the bacteria is enhanced by the substrate as compared, for example, to a corresponding substrate that is free of the any of the metals, boronic acids, nucleic acids, or combinations thereof, disclosed herein.

The source of light can include any suitable light source such as an ultraviolet radiation source, a visible radiation source, an infrared radiation source. The magnifying lens can be attached to a camera. In some embodiments, the camera is a stand-alone camera. Images from the camera can be transmitted for processing through a wire or wireless connection to a computer. In other embodiments, the camera is a component of a computer, tablet, or smartphone.

In operation, the substrate can be used for optical detection of a biological component such as bacteria. A method of using the substrate can include contacting the substrate with an analyte. The analyte can be taken from an animal or a plant. For example, the analyte can include animal tissue, plant tissue, animal extract (e.g., milk), plant extract, or a mixture thereof. The analyte can also be a wash (e.g., a water wash or buffer wash) taken from produce or an article such as furniture or a device such as a computer. The analyte may or may not include a bacteria. The substrate is then exposed to light and one or more images of the substrate are captured. Contacting or incubation can occur at any discrete point or continuously over an amount of time in a range of from about 0.1 hours to about 10 hours, about 1 hour to about 2.5 hours, less than, equal to, or greater than about 0.1 hours, 0.5, 1, 1.5, 2, 2.5, 3, 3.5, 4, 4.5, 5, 5.5, 6, 6.5, 7, 7.5, 8, 8.5, 9, 9.5, or about 10 hours.

During that time, in embodiments including boronic acids, any bacteria that is present bonds to the diol moiety of the boronic acid component. On balance, more bacteria bonds to the boronic acid component than do other biological components. This may be due to the high concentration of polysaccharides in the cell walls of bacteria, which react well with the diol moieties. Some boronic acids may be selective in that that bond to one species of bacteria over another, but in most embodiments, boronic acids are not specific and instead bond to bacteria in a non-selective manner. Conversely, in embodiments that include nucleic acid aptamers, the nucleic acids will tend to only interact and bind with a specific bacteria. The specific interaction between the nucleic acid and the bacteria may be between select nucleotide and surface proteins on the bacteria. Incubation can take place in the presence of the bicarbonate component, a sodium hydroxide composition, a PBS buffer or a combination thereof.

Image capturing can occur at any discrete point or continuously over an amount of time in a range of from instantaneous to about 0.5 hours to about 10 hours, about 1 hour to about 2.5 hours, less than, equal to, or greater than about 0.5, 1, 1.5, 2, 2.5, 3, 3.5, 4, 4.5, 5, 5.5, 6, 6.5, 7, 7.5, 8, 8.5, 9, 9.5, or about 10 hours. In some examples it may be desirable to dry the substrate before imaging.

As the image is captured by a camera, it can be magnified using the magnifying lens. A processor in a phone, tablet, or computer then executes a protocol such as that found in a software program like an application produce an image of the substrate. The presence of bacteria can be confirmed by the presence of contrast on the substrate indicate the presence of bacteria. That is, the presence of a dark region contrasted against a comparatively light background of the substrate positively indicates that the bacteria is present in the biological sample. The dark region can be present as a spot or another geometric shape. Thus, the presence of a bacteria can be qualitatively confirmed. In some embodiments, a pixel intensity of the dark region can be measured and used to calculate the concentration of the bacteria.

### EXAMPLES

Various embodiments of the present disclosure can be better understood by reference to the following Examples which are offered by way of illustration. The present disclosure is not limited to the Examples given herein.

### Example 1 Preparation of the Substrate

A gold coated slide (Thermo Scientific Madison, Wis.) was washed with deionized water and cut into rectangular pieces (approximately 2.5 by 3.5 mm) using a glass knife and cutter to form substrate precursors. The substrate precursors were then washed with ethanol (Pharmco-aaper Chicago, Ill.) for 30 seconds and placed into 4 mL of 1 mM 3-mercaptophenylboronic acid (3-MPBA)-ethanol (AstaTech Bristol, Pa.) solution in a sterile 5 mL test tube. The test tube was put onto a shaker (speed=20 RPM) for approximately 17 hours. After 24 hours, the substrate was formed and was washed with ethanol for 20 seconds and put into a 96-well plate. 150 μL of ethanol was pipetted into each well to stabilize the substrate until use (approximately 1 hour).

### Example 2 Bacterial Detection

Water, buffering chemicals (50 mM ammonia bicarbonate), and the biological sample, were added into the vial including the substrate. The mixture was incubated for 30 minutes. The substrate was removed and rinsed with water. A smartphone including a magnification lens was positioned over the substrate and a photograph was taken.

### Example 3: Results

### Example 3.1 Using a High Precision Lab Microscopy

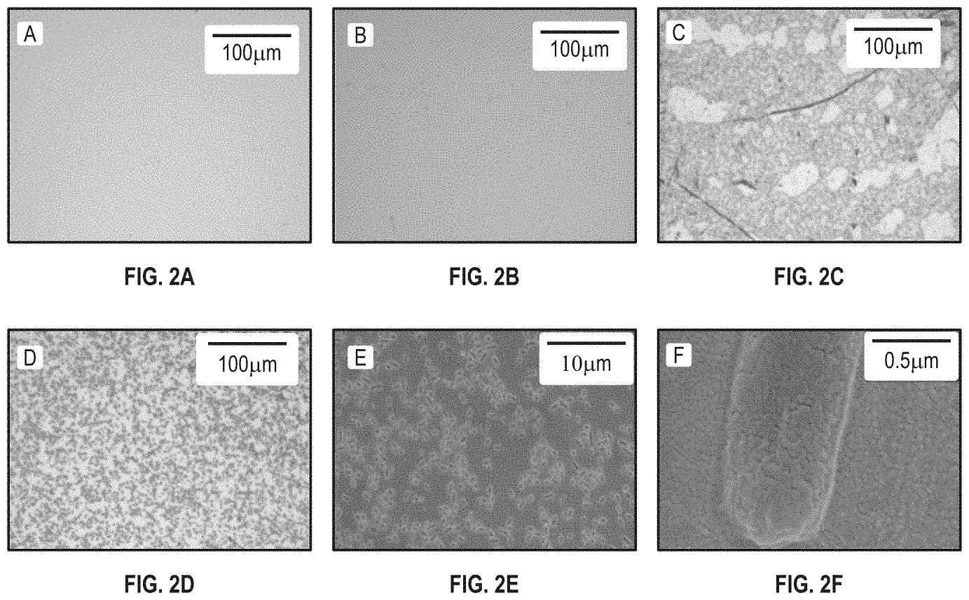

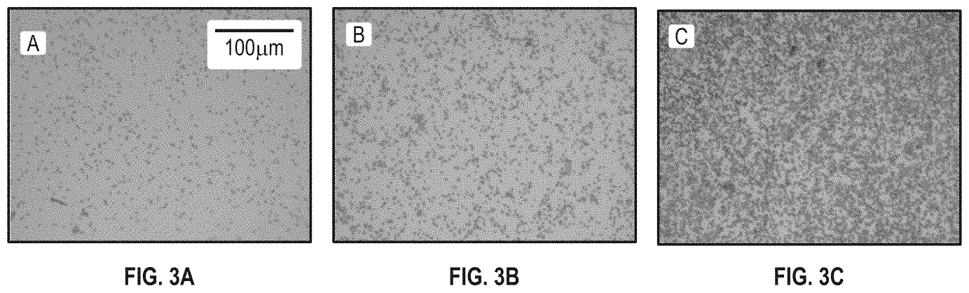

FIG. 2 shows that after bacteria cells bound to the 3-MPBA coated substrates, the bacterial cells were clearly visible under the 20× objective lens (FIG. 2D). Those black spots were confirmed as individual bacterial cells using scanning electron microscope (FIGS. 2E and 2F). When the substrates are free of coating with 3-MPBA, there were no clear black spots.

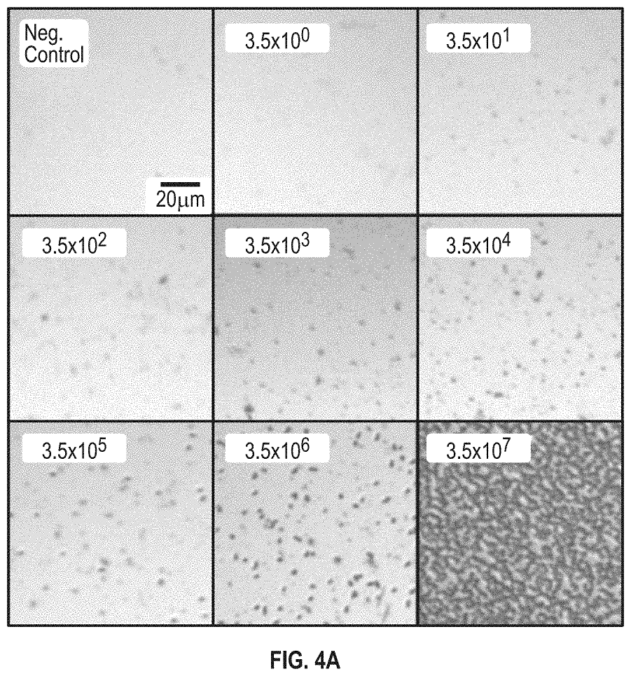

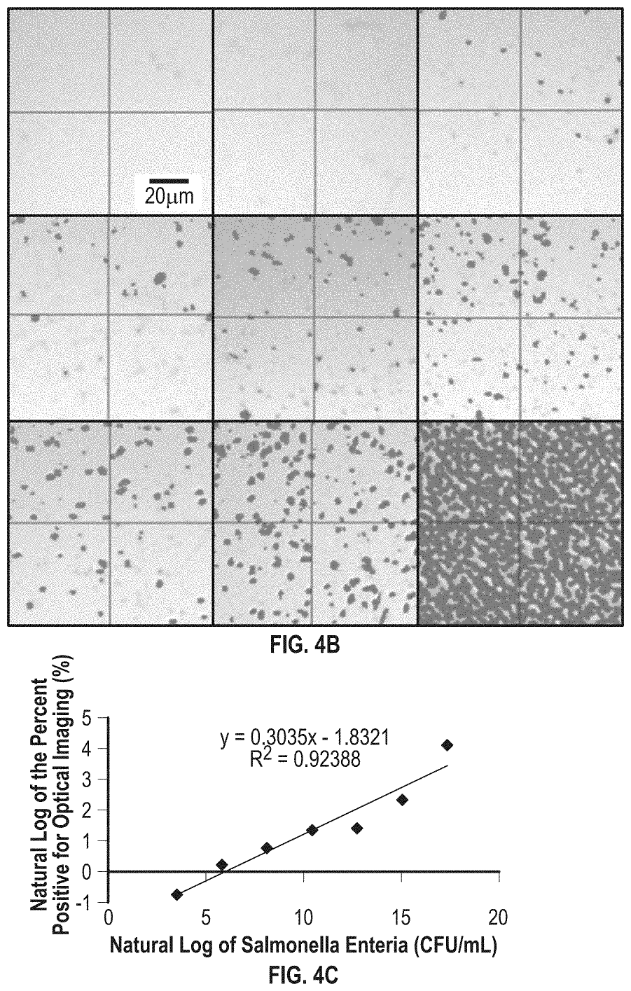

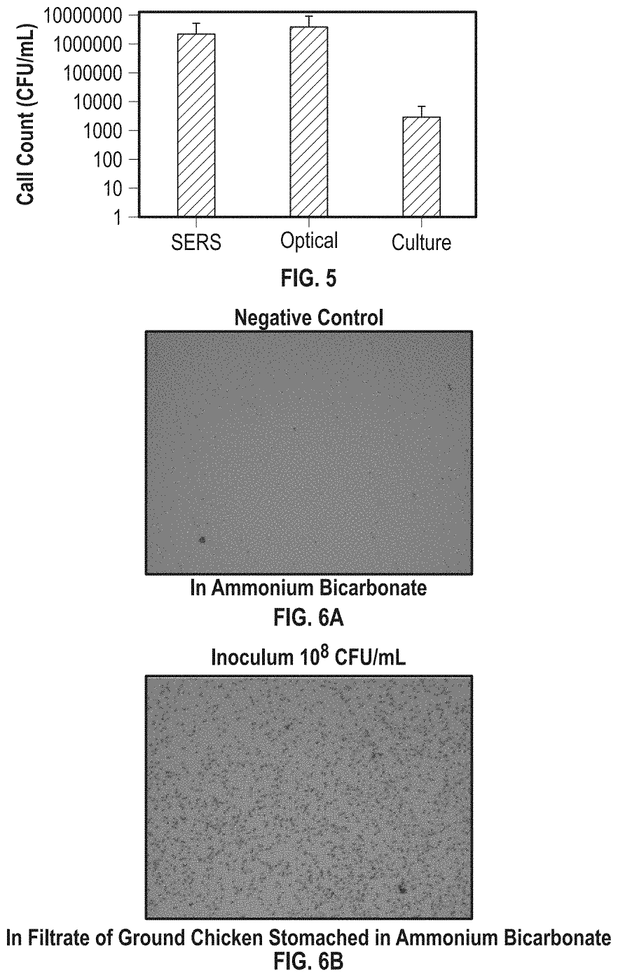

Ammonia bicarbonate was found to be helpful in this Example. As shown in FIG. 4A, bacteria suspended in water were not captured effectively compared to sodium hydroxide (FIG. 4B) and ammonia bicarbonate (both of the pH was 8.4) (FIG. 4C). This is because the boronic acid-diol reaction is more favorable in the alkaline condition. There also appears to be a noticeabble difference between sodium hydroxide and ammonia bicarbonate at the same pH. This demonstrates that ammonia bicarbonate has the ability to enhance the boronic acid-diol reaction more than just providing the alkaline condition. It may also function as protecting 3-MPBA from degradation due to its partial solubility in aqueous mediums, protecting bacteria from lysing due to an increased osmotic pressure effect, enhancing the boronic acid-bacteria interaction by causing boronic acid to be positively charged thus increasing binding affinity to negatively charged bacteria. The optical properties of the visually enhanced bacteria were probed and it was found that these bacteria absorb less light in both the infrared and UV-visible light regions than untreated bacteria.

FIG. 4A shows the raw optical images of bacteria of different concentration. After the pixel intensity analysis (FIG. 4B), a power function has been chosen to model the data. Power functions present a suitable mathematical function for modeling surface area of biological species. This is primarily due to the fact that biological organisms are generally shaped as right cylinders. The mathematical equation for the surface area of a cylinder shape is: A=1/2(2πrh+2πr²). Thus, the equation of a power function, ƒ(x)=kx^{a}, presents a suitable choice. The data is then transformed taking the natural log of both the x and y axis which generates a fairly linear relationship (FIG. 4C) with an R² value of 0.93504. The linearity of the transformed data demonstrates that the optical imaging has the ability to be used for preliminary quantification.

The method to detect bacteria was tested in pond water which was collected at University of Massachusetts, Amherst. The pond water was adjusted by adding ammonia bicarbonate powder to final concentration of 50 mM. Then, 100 μL of the water was added onto the substrate within the 96 well-plate and incubated for 1 hour. The substrate was then taken out of the well plate and dried for optical analysis using the light microscope with 20× objective lens. The optical results showed that there were ˜5×10⁶ CFU/mL bacteria cells in the pond water. To compare the result of this optical method with other methods, two additional tests on the same water sample. Surface enhanced Raman spectroscopy (SERS) was used to confirm the optical detection. SERS is a technique that measures chemical signature of the analyte on the surface of a nano-substrate. To detect bacteria captured on the substrate, a sandwich assay was established by adding 3-MPBA and silver nanoparticles to enhance the signal of captured bacteria. The SERS result showed similar bacteria counts in comparison to the optical method. Additionally, a culturing method (TSA, 37° C., 48 hours) was used to plate count the bacteria in pond water. The result showed much less bacterial counts using the plate count method. The optical and SERS methods are based on the physical or chemical properties of single bacterial cells rather than the ability of the cells to grow into colonies. Therefore, both optical and SERS methods may detect non-culturable bacteria cells as long as their cell wall remain intact. This data demonstrates the high sensitivity and reliability of the optical method to detect bacteria. The method was also used to detect bacteria inoculated Salmonella in ground chicken. The result shows we were able to detect Salmonella in filtrate of ground chicken stomached in ammonium bicarbonate. FIG. 6A shows a negative control and FIG. 6B shows Salmonella detected therein. This protocol could be applied to detect bacteria in many other meats, or even in vegetables such as spinach.

### Example 3.2 Using a Smartphone Attached to a Smartphone Microscope Adapter

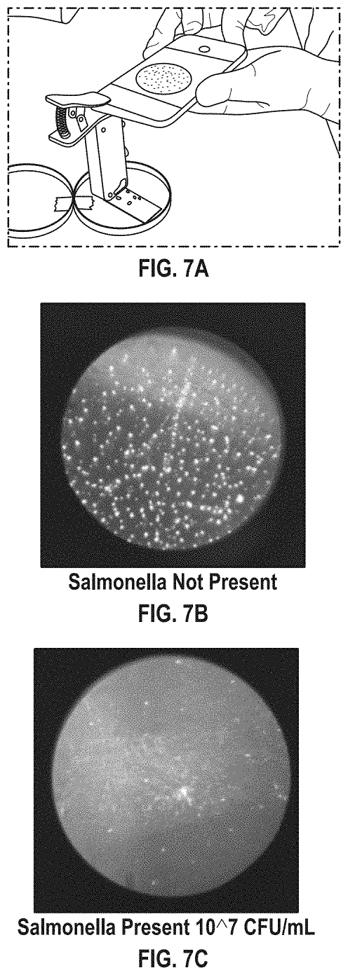

Additionally, a smartphone (iPhone 7, Apple Inc.) and a cellphone microscopic adaptor (200× optical zoom, XFox, $20 from Amazon) was used to take photos or capture continuous images of captured bacteria. The microscopic adaptor had a built-in clip which attached onto the phone to take the photo (FIG. 7A). As shown in FIGS. 7B and 7C, the bacteria cells appeared as bright spots on the grey background. The difference between images as compared to those from the optical microscope may be attributable to the different light settings. The photos taken by the smartphone were under white LED light inside the adaptor. In the control, there were also some light spots that may be due to the contamination of the substrate or artifacts of the photos that would result in false positive. This data demonstrates the feasibility of using a smartphone to detect bacteria.

### Example 4 Aptamer-Coated Substrate

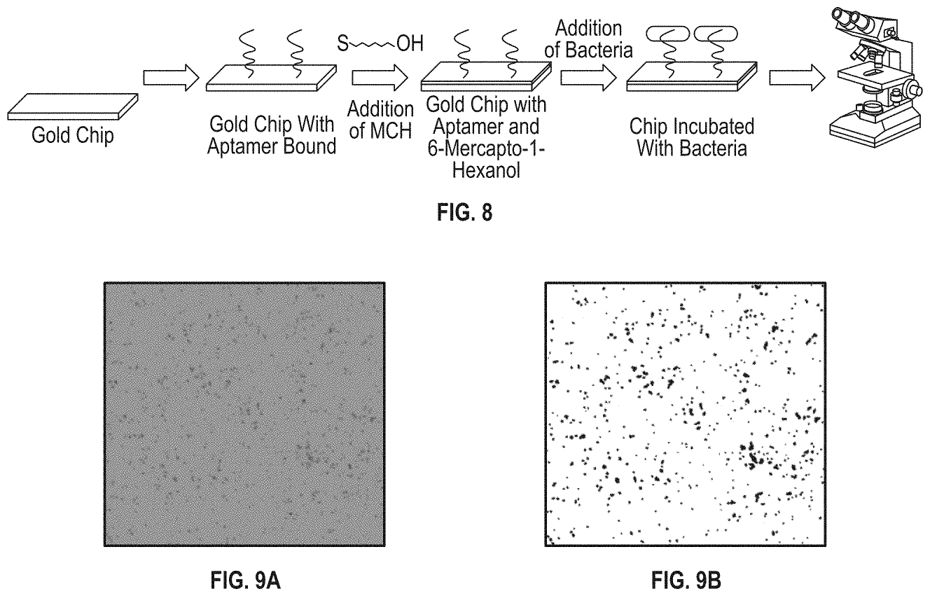

FIG. 8 schematically shows a method of making an aptamer coated gold chip or substrate and a method of using the same to selectively detect a predetermined bacteria such as Salmonella or Listeria.

The aptamers included thiolated sequences with about 5 to about 10 additional repeated nucleotides (e.g. A or T) added at the 5′ end as spacers. The sequences were used to specifically interact with and detect predetermined bacteria species. The sequences corresponded to SEQ ID 1 and SEQ ID 2 and are reproduced below:

1. Salmonella detection using aptamer:
5′-S-S-AAA AAA CTC CTC TGA CTG TAA CCA CGC ACA AAG
GCT CGC GCA TGG TGT GTA CGT TCT TAC AGA GGT-3′
2. Listeria detection using aptamer:
5′-S-S-TTT TTT TTT TAT CCATGG GGC GGA GAT GAG GGG
GAG GAG GGC GGG TAC CCG GTT GAT-3′

Before the aptamer was used, the aptamer was heated at 96° C. for 3 minutes in a thermal cycler and cooled down at room temperature (about 25° C.). This process helped the aptamer to form a functional 3D structure. Following heating, tris(2-carboxyethyl)phosphine (TCEP) was added to cleave the disulfide bond to thiol for a better interaction with the gold coated chip or substrate. The prepared aptamer was then incubated with the gold chip for about 5 to about 24 hours in phosphate-buffered saline (PBS) buffer, which included additional Mg²⁺ and Ca²⁺ in a concentration ranging from about of 0.2-2 mM. The chip was washed using buffer after the incubation to remove any unbound aptamer.

Following washing, the gold coated chip was incubated with 6-mercapto-1-hexanol for a time ranging from about 30 minutes to about 16 hours. This helped to backfill the spaces and prevent the aptamers from laying down on the chips. The chips were then washed with water and were ready for bacteria incubation.

One mL bacteria having variable concentrations was incubated with the substrate for a time in a range of from about 30 minutes to about 2 hours. After the incubation, the chips were rinsed with the PBS buffer followed by water. The substrate was then dried and examined under a microscope. Images collected with the microscope were analyzed with Image J software, which transferred the image into a binary image including dots. FIG. 9A shows the image from the microscope and FIG. 9B, shows the transferred binary image. The dots (bacteria) were counted as a measure for quantification.

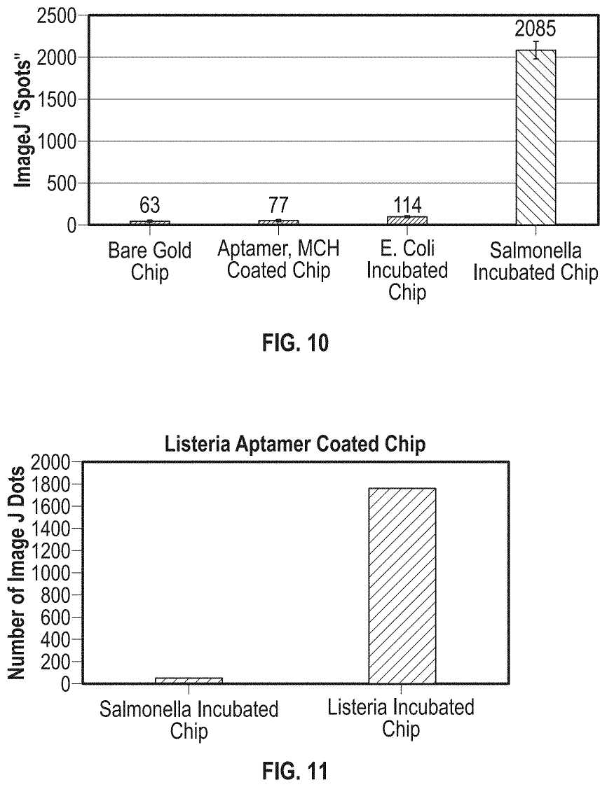

The substrates of Example 4 showed great selectivity for binding to a predetermined bacteria as a function of the aptamer that was functionalized to the gold chip. FIG. 10 is a bar graph showing the number of Image J spots or dots recorded on a bare gold (e.g., no aptamer) chip or substrate that was not incubated with bacteria; a chip including MCH, and the aptamer of SEQ ID 1, but not incubated with bacteria; the chip including MCH, and the aptamer of SEQ ID 1, incubated with Lysteria; and the chip including MCH, and the aptamer of SEQ ID 1, incubated with Salmonella. As shown, Salmonella was selectively captured over Listeria by virtue of the aptamer of SEQ ID 1.

FIG. 11 is a bar graph showing the number of Image J spots or dots recorded on the gold chip or substrate including MCH, and the aptamer of SEQ ID 2, incubated with Lysteria; and the chip including MCH, and the aptamer of SEQ ID 1, incubated with Salmonella. As shown, Listeria was selectively captured over Salmonella by virtue of the aptamer of SEQ ID 1.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present disclosure. Thus, it should be understood that although the present disclosure has been specifically disclosed by specific embodiments and optional features, modification and variation of the concepts herein disclosed may be resorted to by those of ordinary skill in the art, and that such modifications and variations are considered to be within the scope of embodiments of the present disclosure.

### Additional Embodiments

The following exemplary embodiments are provided, the numbering of which is not to be construed as designating levels of importance:

Embodiment 1 provides a substrate comprising:
a metallic layer; and
a boronic acid component at least partially coating the metallic layer.

Embodiment 2 provides the substrate of Embodiment 1, wherein the metallic layer comprises a semi-conductive metal.

Embodiment 3 provides the substrate of Embodiment 1, wherein the metallic layer comprises Ag₂O, elemental silver, elemental gold, elemental copper, elemental platinum, mixtures thereof, alloys thereof, or combinations thereof.

Embodiment 4 provides the substrate of any one of Embodiments 1-3, wherein the metallic layer comprises a continuous layer, a collection of microparticles, nanoparticles, or mixtures thereof.

Embodiment 5 provides the substrate of Embodiment 4, wherein at least one of the microparticles and the nanoparticles comprise a substantially spherical shape.

Embodiment 6 provides the substrate of Embodiment 3, wherein the continuous layer has a substantially uniform thickness.

Embodiment 7 provides the substrate of any one of Embodiments 1-6, wherein a surface of the metallic layer is substantially smooth.

Embodiment 8 provides the substrate of any one of Embodiments 1-7, wherein the metallic layer reflects about 50% to about 99% of light to which it is exposed.

Embodiment 9 provides the substrate of any one of Embodiments 1-8, wherein the metallic layer reflects about 80% to about 99% of light to which it is exposed.

Embodiment 10 provides the substrate of any one of Embodiments 1-9, wherein the metallic layer comprises elemental gold.

Embodiment 11 provides the substrate of Embodiment 1, further comprising a polymeric layer attached to the metallic layer.

Embodiment 12 provides the substrate of Embodiment 11, wherein the polymeric layer comprises polyvinylidene fluoride, polyethylene, polypropylene, polyvinyl chloride, polystyrene, polyamide, polytetrfluoroethylene, thermoplastic polyurethane, copolymers thereof, or mixtures thereof.

Embodiment 13 provides the substrate of any one of Embodiments 1-12, wherein the boronic acid component is an alkyl boronic acid, an alkenyl boronic acid, a heteroaryl boronic acid, a salt thereof, or a mixture thereof.

Embodiment 14 provides the substrate of Embodiment 13, wherein the aryl boronic acid is an unsubstituted aryl boronic acid, a monosubstituted aryl boronic acid, a disubstituted aryl bornonic acid, a trisubstituted aryl boronic acid, a tetrasubstitued aryl boronic acid, a pentasubstituted aryl boronic acid, a salt thereof, or a mixture thereof.

Embodiment 15 provides the substrate of any one of Embodiments 1-14, wherein the boronic acid component is 2-bromophenylboronic acid, 3-bromophenylboronic acid, 4-bromophenylboronic acid, 2-chlorophenylboronic acid, 3-chlorophenylboronic acid, 4-chlorophenylboronic acid, 2-fluorophenylboronic acid, 3-fluorophenylboronic acid, 4-fluorophenylboronic acid, 2-iodophenylboronic acid, 3-iodophenylboronic acid, 4-iodophenylboronic acid, 2-nitrophenylboronic acid, 3-nitrophenylboronic acid, 4-nitrophenylboronic acid, 3-mercaptophenylboronic acid, 4-mercaptophenylboronic acid, 2-hydroxyphenylboronic acid, 3-hydroxyphenylboronic acid, 4-hydroxyphenylboronic acid, 3-aminophenylboronic acid, (4-aminosulfonylphenyl)boronic acid, 3-boronobenzenesulfonamide, benzene-1,4-diboronic acid, (4-chlorocarbonylphenyl)boronic anhydride, 2,2-difluoro-benzo[1,3]dioxole-5-boronic acid, 2-(trifluoromethyl)phenylboronic acid, 3-(trifluoromethyl)phenylboronic acid, 4-(trifluoromethyl)phenylboronic acid, 2-(trifluoromethoxy)phenylboronic acid, 3-(trifluoromethoxy)phenylboronic acid, 4-(trifluoromethoxy)phenylboronic acid, 2-cyanophenylboronic acid, 3-cyanophenylboronic acid, 4-cyanophenylboronic acid, 2-formylphenylboronic acid, 3-formylphenylboronic acid, 4-formylphenylboronic acid, 2-(bromomethyl)phenylboronic acid, 3-(bromomethyl)phenylboronic acid, 4-(bromomethyl)phenylboronic acid, 3-boronobenzothioamide, 2-aminocarbonylphenylboronic acid, 3-aminocarbonylphenylboronic acid, 4-aminocarbonylphenylboronic acid, 2-(methylthio)phenylboronic acid, 3-(methylthio)phenylboronic acid, 4-(methylthio)phenylboronic acid, 2-(methylsulfonyl)phenylboronic acid, 3-(methylsulfonyl)phenylboronic acid, 4-(methylsulfonyl)phenylboronic acid, N-4-methanesulfonamidephenylboronic acid, 4-(cyanomethyl)benzeneboronic acid, 2-methoxycarbonylphenylboronic acid, 3-methoxycarbonylphenylboronic acid, 3-methoxycarbonylphenylboronic acid, 2-acetamidophenylboronic acid, 3-acetamidophenylboronic acid, 4-acetamidophenylboronic acid, 3-ethylsulfinylphenylboronic acid, 4-ethylsulfinylphenylboronic acid, or mixtures thereof.

Embodiment 16 provides the substrate of any one of Embodiments 1-15, wherein the boronic acid component is a structure according to any of Formula (I), Formula (II), a salt thereof, or mixtures thereof:

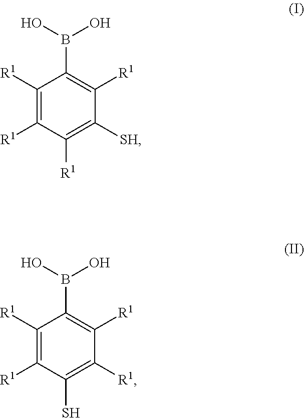

wherein at each occurrence, R¹ is independently chosen from —H, —OH, —NH₂, substituted or unsubstituted (C₁-C₂₀)hydrocarbyl, and combinations thereof.

Embodiment 17 provides the substrate of Embodiment 16, wherein at each occurrence the substituted or unsubstituted (C₁-C₂₀)hydrocarbyl is chosen from (C₁-C₂₀)alkyl, (C₁-C₂₀)alkenyl, (C₁-C₂₀)alkynyl, (C₁-C₂₀)acyl, (C₁-C₂₀)cycloalkyl, (C₁-C₂₀)aryl, and (C₁-C₂₀)alkoxy, and combinations thereof.

Embodiment 18 provides the substrate of any one of Embodiments 16 or 17, wherein at each occurrence R¹ is —H.

Embodiment 19 provides the substrate of any one of Embodiments 1-18, wherein the substrate is at least partially disposed in an aqueous solution.

Embodiment 20 provides the substrate of Embodiment 19, wherein the aqueous solution comprises a bicarbonate component.

Embodiment 21 provides the substrate of Embodiment 20, wherein the bicarbonate component comprises sodium bicarbonate, potassium bicarbonate, cesium bicarbonate, magnesium bicarbonate, calcium bicarbonate, ammonium bicarbonate, or mixtures thereof.

Embodiment 22 provides the substrate of any one of Embodiments 20 or 21, wherein the bicarbonate component is ammonium bicarbonate.

Embodiment 23 provides the substrate of any one of Embodiments 20-22, wherein a concentration of the bicarbonate component in the aqueous solution is in a range of from about 1 mM to about 100 mM.

Embodiment 24 provides the substrate of any one of Embodiments 20-23, wherein a concentration of the bicarbonate component in the aqueous solution is in a range of from about 40 mM to about 60 mM.

Embodiment 25 provides the substrate of any one of Embodiments 19-24, wherein a pH of the aqueous solution is in a range of from about 7 to about 12.

Embodiment 26 provides the substrate of any one of Embodiments 19-25, wherein a pH of the aqueous solution is in a range of from about 8 to about 10.

Embodiment 27 provides the substrate of any one of Embodiments 1-26, further comprising a chamber at least partially bounding the substrate.

Embodiment 28 provides the substrate of Embodiment 20, wherein the chamber is a well.

Embodiment 29 provides the substrate of any one of Embodiments 19-28, wherein the aqueous solution further comprises a biological component.

Embodiment 30 provides the substrate of Embodiment 29, wherein the biological component comprises bacteria.

Embodiment 31 provides the substrate of Embodiment 30, wherein the bacteria comprises a gram-positive bacteria, a gram-negative bacteria, or a mixture thereof.

Embodiment 32 provides the substrate of any one of Embodiments 29 or 31, wherein the bacteria comprises Clostridium botulinum, Listeria monocytogenes, Acetic acid bacteria, Acidaminococcus, Acinetobacter baumannii, Agrobacterium tumefaciens, Akkermansia muciniphila, Anaerobiospirillum, Anaerolinea thermolimosa, Anaerolinea thermophila, Arcobacter, Arcobacter skirrowii, Armatimonas rosea, Azotobacter salinestris, Bacteroides, Bacteroides fragilis, Bacteroides ureolyticus, Bacteroidetes, Bartonella japonica, Bartonella koehlerae, Bartonella taylorii, Bdellovibrio, Brachyspira, Bradyrhizobium japonicum, Caldilinea aerophile, Cardiobacterium hominis, Chaperone-Usher fimbriae, Christensenella, Chthonomonas calidirosea, Coxiella burnetiid, Cyanobacteria, Cytophaga, Dehalogenimonas lykanthroporepellens, Desulfurobacterium atlanticum, Devosia pacifica, Devosia psychrophila, Devosia soli, Devosia subaequoris, Devosia submarina, Devosia yakushimensis, Dialister, Dictyoglomus thermophilum, Enterobacter, Enterobacter cloacae, Enterobacter cowanii, Enterobacteriaceae, Enterobacteriales, Escherichia, Escherichia coli, Escherichiafergusonii, Escherichia hermannii, Fimbriimonas ginsengisoli, Flavobacterium, Flavobacterium akiainvivens, Francisella novicida, Fusobacterium necrophorum, Fusobacterium nucleatum, Fusobacterium polymorphum, Haemophilus felis, Haemophilus haemolyticus, Haemophilus influenzae, Haemophilus pittmaniae, Helicobacter, Kingella kingae, Klebsiella pneumoniae, Kluyvera ascorbate, Kluyvera cryocrescens, Legionella, Legionella clemsonensis, Legionella pneumophila, Leptonema illini, Leptotrichia buccalis, Levilinea saccharolytica, Luteimonas aquatic, Luteimonas composti, Luteimonas lutimaris, Luteimonas marina, Luteimonas mephitis, Luteimonas vadose, Megamonas, Megasphaera, Meiothermus, Meiothermus timidus, Methylobacterium fiujisawaense, Morax-Axenfeld diplobacilli, Moraxella, Moraxella bovis, Moraxella osloensis, Morganella morganii, Mycoplasma spumans, Neisseria cinereal, Neisseria gonorrhoeae, Neisseria meningitidis, Neisseria polysaccharea, Neisseria sicca, Nitrosomonas eutropha, Nitrosomonas halophila, Nonpathogenic organisms, OMPdb, Pectinatus, Pedobacter heparinus, Pelosinus, Propionispora, Proteobacteria, Proteus mirabilis, Proteus penneri, Pseudomonas, Pseudomonas aeruginosa, Pseudomonas luteola, Pseudoxanthomonas broegbernensis, Pseudoxanthomonas japonensis, Rickettsia rickettsia, Salinibacter ruber, Salmonella, Salmonella bongori, Salmonella enterica, Samsonia, Selenomonadales, Serratia marcescens, Shigella, Shimwellia, Solobacterium moorei, Sorangium cellulosum, Sphaerotilus natans, Sphingomonas gei, Spirochaeta, Spirochaetaceae, Sporomusa, Stenotrophomonas, Stenotrophomonas nitritireducens, Thermotoga neapolitana, Thorselliaceae, Trimeric autotransporter adhesion, Vampirococcus, Verminephrobacter, Vibrio adaptatus, Vibrio azasii, Vibrio campbellii, Vibrio cholerae, Victivallis vadensis, Vitreoscilla, Wolbachia, Yersiniaceae, Zymophilus, strains thereof, or combinations thereof.

Embodiment 33 provides a method of making the substrate of any one of Embodiments 1-32, the method comprising coating the metallic layer with the boronic acid component.

Embodiment 34 provides the method of Embodiment 33, further comprising washing the metallic layer with water.

Embodiment 35 provides the method of any one of Embodiments 33 or 34, further comprising washing the metallic layer with an organic solvent.

Embodiment 36 provides the method of any one of Embodiments 33-35, wherein coating the metallic layer comprises contacting the metallic layer with a solution comprising the boronic acid component.

Embodiment 37 provides the method of Embodiment 36, wherein a concentration of the boronic acid component in the solution is in a range of from about 0.5 mM to about 5 mM.

Embodiment 38 provides the method of any one of Embodiments 36 or 37, wherein a concentration of the boronic acid component in the solution is in a range of from about 0.5 mM to about 1.5 mM.

Embodiment 39 provides the method of any one of Embodiments 33-38 wherein coating occurs over a time ranging from about 1 hour to about 48 hours.

Embodiment 40 provides the method of any one of Embodiments 33-39 wherein coating occurs over a time ranging from about 10 hours to about 20 hours.

Embodiment 41 provides the method of any one of Embodiments 33-40, further comprising disposing the substrate in a well.

Embodiment 42 provides the method of any one of Embodiments 33-41, further comprising disposing the substrate in an aqueous medium.

Embodiment 43 provides the method of any one of Embodiments 33-42, further comprising adjusting the pH to a value in a range of from about 7 to about 12.

Embodiment 44 provides the method of any one of Embodiments 33-43, further comprising adjusting the pH to a value in a range of from about 8 to about 10.

Embodiment 45 provides the method of any one of Embodiments 33-44, further comprising contacting the substrate with the bicarbonate component.

Embodiment 46 provides a system for detection of a biological component, the system comprising:
the substrate according to any one of Embodiments 1-32 or formed according to any one of Embodiments 33-45; and a magnifying lens in optical communication with the substrate.

Embodiment 47 provides the system of Embodiment 46, wherein the magnifying lens has a magnification range of from about 10× to about 100×.

Embodiment 48 provides the system of any one of Embodiments 46 or 47, wherein the magnifying lens has a magnification range of from about 15× to about 25×.

Embodiment 49 provides the system of any one of Embodiments 46-48, wherein the magnifying lens is attached to a camera.

Embodiment 50 provides the system of any one of Embodiments 46-49, wherein the camera is a component of a computer, smartphone, or tablet.

Embodiment 51 provides the system of any one of Embodiments 46-50, wherein the substrate is disposed in a sealed container.

Embodiment 52 provides the system of Embodiment 51, wherein the sealed container comprises a first chamber containing the substrate and a second chamber containing the bicarbonate component.

Embodiment 53 provides a method of using the substrate according to any one of Embodiments 1-32 or formed according to any one of Embodiments 33-45, or using the system of any one of Embodiments 46-52, the method comprising:
contacting the substrate with an analyte;
exposing the substrate to light;
capturing an image of the substrate; and
visually confirming the presence or absence of bacteria.

Embodiment 54 provides the method of Embodiment 53, further comprising magnifying the substrate using a magnifying lens.

Embodiment 55 provides the method of any one of Embodiments 53 or 54, wherein the analyte comprises animal tissue, plant tissue, animal extract, plant extract, or a mixture thereof.

Embodiment 56 provides the method of any one of Embodiments 53-55, wherein the analyte comprises bacteria.

Embodiment 57 provides the method of any one of Embodiments 53-56, wherein the image is captured over an amount of time in a range of from about 0.5 hours to about 10 hours.

Embodiment 58 provides the method of any one of Embodiments 53-57, wherein the image is captured over an amount of time in a range of from about 1 hour to about 2.5 hours.

Embodiment 59 provides the method of any one of Embodiments 53, wherein the presence of contrast on the substrate indicate the presence of bacteria.

Embodiment 60 provides the method of Embodiment 59, wherein the bacteria appears as a dark region contrasted against a light background of the substrate.

Embodiment 61 provides the method of Embodiment 60, further comprising measuring a pixel intensity of the dark spot and determining a concentration of the bacteria.

Embodiment 62 provides the method of any one of Embodiments 53-61, further comprising contacting the substrate with the bicarbonate component.

Embodiment 63 provides the method of Embodiment 62, wherein contacting the substrate with the bicarbonate component comprises releasing at least one of the substrate and the bicarbonate component from a sealed chamber.

Embodiment 64 provides the method of any one of Embodiments 53-63, wherein contacting comprises incubating the analyte and the substrate.

Embodiment 65 provides the method of Embodiment 64, further comprising drying the substrate following incubation but before exposing the substrate to light.

Embodiment 66 provides a substrate comprising:
a metallic layer; and
a nucleic acid aptamer component functionalized with the metallic layer.

Embodiment 67 provides the substrate of Embodiment 66, wherein the metallic layer comprises a semi-conductive metal.

Embodiment 68 provides the substrate of Embodiment 66, wherein the metallic layer comprises Ag₂O, elemental silver, elemental gold, elemental copper, elemental platinum, mixtures thereof, alloys thereof, or combinations thereof.

Embodiment 69 provides the substrate of any one of Embodiments 66-68, wherein the metallic layer comprises a continuous layer, a collection of microparticles, nanoparticles, or mixtures thereof.

Embodiment 70 provides the substrate of Embodiment 69, wherein at least one of the microparticles and the nanoparticles comprise a substantially spherical shape.

Embodiment 71 provides the substrate of Embodiment 68, wherein the continuous layer has a substantially uniform thickness.

Embodiment 72 provides the substrate of any one of Embodiments 66-71, wherein a surface of the metallic layer is substantially smooth.

Embodiment 73 provides the substrate of any one of Embodiments 66-72, wherein the metallic layer reflects about 50% to about 99% of light to which it is exposed.

Embodiment 74 provides the substrate of any one of Embodiments 66-73, wherein the metallic layer reflects about 80% to about 99% of light to which it is exposed.

Embodiment 75 provides the substrate of any one of Embodiments 66-74, wherein the metallic layer comprises elemental gold.

Embodiment 76 provides the substrate of Embodiment 66, further comprising a substrate coating layer attached to the metallic layer.

Embodiment 77 provides the substrate of Embodiment 76, wherein the substrate coating layer comprises 6-mercapto-1-hexanol, polyvinylidene fluoride, polyethylene, polypropylene, polyvinyl chloride, polystyrene, polyamide, polytetrfluoroethylene, thermoplastic polyurethane, copolymers thereof, or mixtures thereof.

Embodiment 78 provides the substrate of any one of Embodiments 66-77, wherein the nucleic acid aptamer component comprises deoxyribonucleic acid, ribonucleic acid, or a mixture thereof.

Embodiment 79 provides the substrate of Embodiment 78, wherein the deoxyribonucleic acid, ribonucleic acid or the mixture thereof comprises single stranded deoxyribonucleic acid, single stranded ribonucleic acid, or a mixture thereof.

Embodiment 80 provides the substrate of any one of Embodiments 66-79, wherein the aptamer comprises the sequence according to SEQ ID 1.

Embodiment 81 provides the substrate of any one of Embodiments 66-80, wherein the aptamer comprises the sequence according to SEQ ID 2.

Embodiment 82 provides the substrate of Embodiment 81, wherein the aptamer is configured to bind to a predetermined bacteria.

Embodiment 83 provides the substrate of any one of Embodiments 66-82, wherein the substrate is at least partially disposed in an aqueous solution.

Embodiment 84 provides the substrate of Embodiment 83, wherein the aqueous solution comprises a PBS buffer.

Embodiment 85 provides the substrate of Embodiment 84, wherein the PBS buffer comprises PBS diluted in water at a ratio of about 1:1.

Embodiment 86 provides the substrate of any one of Embodiments 83-85, wherein a pH of the aqueous solution is in a range of from about 7 to about 12.

Embodiment 87 provides the substrate of any one of Embodiments 83-86, wherein a pH of the aqueous solution is in a range of from about 8 to about 10.

Embodiment 88 provides the substrate of any one of Embodiments 66-87, further comprising a chamber at least partially bounding the substrate.

Embodiment 89 provides the substrate of Embodiment 84, wherein the chamber is a well.

Embodiment 90 provides the substrate of any one of Embodiments 83-89, wherein the aqueous solution further comprises a biological component.

Embodiment 91 provides the substrate of Embodiment 90, wherein the biological component comprises bacteria.

Embodiment 92 provides the substrate of Embodiment 91, wherein the bacteria comprises a gram-positive bacteria, a gram-negative bacteria, or a mixture thereof.

Embodiment 93 provides the substrate of any one of Embodiments 91 or 3192, wherein the bacteria comprises Clostridium botulinum, Listeria monocytogenes, Acetic acid bacteria, Acidaminococcus, Acinetobacter baumannii, Agrobacterium tumefaciens, Akkermansia muciniphila, Anaerobiospirillum, Anaerolinea thermolimosa, Anaerolinea thermophila, Arcobacter, Arcobacter skirrowii, Armatimonas rosea, Azotobacter salinestris, Bacteroides, Bacteroides fragilis, Bacteroides ureolyticus, Bacteroidetes, Bartonella japonica, Bartonella koehlerae, Bartonella taylorii, Bdellovibrio, Brachyspira, Bradyrhizobium japonicum, Caldilinea aerophile, Cardiobacterium hominis, Chaperone-Usher fimbriae, Christensenella, Chthonomonas calidirosea, Coxiella burnetiid, Cyanobacteria, Cytophaga, Dehalogenimonas lykanthroporepellens, Desulfurobacterium atlanticum, Devosia pacifica, Devosia psychrophila, Devosia soli, Devosia subaequoris, Devosia submarina, Devosia yakushimensis, Dialister, Dictyoglomus thermophilum, Enterobacter, Enterobacter cloacae, Enterobacter cowanii, Enterobacteriaceae, Enterobacteriales, Escherichia, Escherichia coli, Escherichia fergusonii, Escherichia hermannii, Fimbriimonas ginsengisoli, Flavobacterium, Flavobacterium akiainvivens, Francisella novicida, Fusobacterium necrophorum, Fusobacterium nucleatum, Fusobacterium polymorphum, Haemophilus felis, Haemophilus haemolyticus, Haemophilus influenzae, Haemophilus pittmaniae, Helicobacter, Kingella kingae, Klebsiella pneumoniae, Kluyvera ascorbate, Kluyvera cryocrescens, Legionella, Legionella clemsonensis, Legionella pneumophila, Leptonema illini, Leptotrichia buccalis, Levilinea saccharolytica, Luteimonas aquatic, Luteimonas composti, Luteimonas lutimaris, Luteimonas marina, Luteimonas mephitis, Luteimonas vadose, Megamonas, Megasphaera, Meiothermus, Meiothermus timidus, Methylobacterium fujisawaense, Morax-Axenfeld diplobacilli, Moraxella, Moraxella bovis, Moraxella osloensis, Morganella morganii, Mycoplasma spumans, Neisseria cinereal, Neisseria gonorrhoeae, Neisseria meningitidis, Neisseria polysaccharea, Neisseria sicca, Nitrosomonas eutropha, Nitrosomonas halophila, Nonpathogenic organisms, OMPdb, Pectinatus, Pedobacter heparinus, Pelosinus, Propionispora, Proteobacteria, Proteus mirabilis, Proteus penneri, Pseudomonas, Pseudomonas aeruginosa, Pseudomonas luteola, Pseudoxanthomonas broegbernensis, Pseudoxanthomonas japonensis, Rickettsia rickettsia, Salinibacter ruber, Salmonella, Salmonella bongori, Salmonella enterica, Samsonia, Selenomonadales, Serratia marcescens, Shigella, Shimwellia, Solobacterium moorei, Sorangium cellulosum, Sphaerotilus natans, Sphingomonas gei, Spirochaeta, Spirochaetaceae, Sporomusa, Stenotrophomonas, Stenotrophomonas nitritireducens, Thermotoga neapolitana, Thorselliaceae, Trimeric autotransporter adhesion, Vampirococcus, Verminephrobacter, Vibrio adaptatus, Vibrio azasii, Vibrio campbellii, Vibrio cholerae, Victivallis vadensis, Vitreoscilla, Wolbachia, Yersiniaceae, Zymophilus, strains thereof, or combinations thereof.

Embodiment 94 provides the substrate of any one of Embodiments 92 or 93, wherein the bacteria comprises Listeria monocytogenes, Salmonella, Salmonella bongori, Salmonella enterica, or a mixture thereof.

Embodiment 95 provides a method of making the substrate of any one of Embodiments 66-94, the method functionalizing the metallic layer with the aptamer component.

Embodiment 96 provides the method of Embodiment 95, further comprising washing the metallic layer with water.

Embodiment 97 provides the method of any one of Embodiments 95 or 96, further comprising washing the metallic layer with an organic solvent.

Embodiment 98 provides the method of any one of Embodiments 95-97, wherein functionalizing the metallic layer comprises contacting the metallic layer with a solution comprising the aptamer component.

Embodiment 99 provides the method of Embodiment 98, wherein a concentration of the aptamer in the solution is in a range of from about 0.5 mM to about 5 mM.

Embodiment 100 provides the method of any one of Embodiments 98 or 99, wherein a concentration of the aptamer component in the solution is in a range of from about 0.5 mM to about 1.5 mM.

Embodiment 101 provides the method of any one of Embodiments 95-100 wherein functionalizing occurs over a time ranging from about 1 hour to about 48 hours.

Embodiment 102 provides the method of any one of Embodiments 95-101 wherein functionalizing occurs over a time ranging from about 10 hours to about 20 hours.

Embodiment 103 provides the method of any one of Embodiments 95-102, further comprising disposing the substrate in a well.

Embodiment 104 provides the method of any one of Embodiments 95-103, further comprising disposing the substrate in an aqueous medium.

Embodiment 105 provides the method of any one of Embodiments 95-104, further comprising adjusting the pH to a value in a range of from about 7 to about 12.

Embodiment 106 provides the method of any one of Embodiments 95-105, further comprising adjusting the pH to a value in a range of from about 8 to about 10.

Embodiment 107 provides the method of any one of Embodiments 95-106, further comprising contacting the substrate with the PBS buffer.

Embodiment 108 provides the method of any one of claims 95-107, further comprising at least partially coating a surface of the metallic component with the coating component.

Embodiment 109 provides a system for detection of a biological component, the system comprising:
the substrate according to any one of Embodiments 66-94 or formed according to any one of Embodiments 33-45; and
a magnifying lens in optical communication with the substrate.

Embodiment 110 provides the system of Embodiment 109, wherein the magnifying lens has a magnification range of from about 10× to about 100×.

Embodiment 111 provides the system of any one of Embodiments 109 or 110, wherein the magnifying lens has a magnification range of from about 15× to about 25×.

Embodiment 112 provides the system of any one of Embodiments 109-111, wherein the magnifying lens is attached to a camera.

Embodiment 113 provides the system of any one of Embodiments 109-112, wherein the camera is a component of a computer, smartphone, or tablet.

Embodiment 114 provides the system of any one of Embodiments 109-113, wherein the substrate is disposed in a sealed container.

Embodiment 115 provides the system of Embodiment 114, wherein the sealed container comprises a first chamber containing the substrate and a second chamber containing the PBS component.

Embodiment 116 provides a method of using the substrate according to any one of Embodiments 66-94 or formed according to any one of Embodiments 95-107, or using the system of any one of Embodiments 108-115, the method comprising:
contacting the substrate with an analyte;
exposing the substrate to light;
capturing an image of the substrate; and
visually confirming the presence or absence of bacteria.

Embodiment 117 provides the method of Embodiment 116, further comprising magnifying the substrate using a magnifying lens.

Embodiment 118 provides the method of any one of Embodiments 116 or 117, wherein the analyte comprises animal tissue, plant tissue, animal extract, plant extract, or a mixture thereof.

Embodiment 119 provides the method of any one of Embodiments 116-118, wherein the analyte comprises bacteria.

Embodiment 120 provides the method of any one of claims 116-119, wherein the bacteria is a predetermined bacteria.

Embodiment 121 provides the method of any one of claims 116-120, wherein the aptamer bonds with a surface protein of the bacteria.

Embodiment 122 provides the method of any one of claims 116-121, wherein the aptamer selectively bonds with specific bacteria.

Embodiment 123 provides the method of any one of Embodiments 116-122, wherein the image is captured over an amount of time in a range of from about 0.5 hours to about 10 hours.

Embodiment 124 provides the method of any one of Embodiments 116-123, wherein the image is captured over an amount of time in a range of from about 1 hour to about 2.5 hours.

Embodiment 125 provides the method of any one of Embodiments 116-124, wherein the presence of contrast on the substrate indicate the presence of bacteria.

Embodiment 126 provides the method of Embodiment 125, wherein the bacteria appears as a dark region contrasted against a light background of the substrate.

Embodiment 127 provides the method of Embodiment 126, further comprising measuring a pixel intensity of the dark spot and determining a concentration of the bacteria.

Embodiment 128 provides the method of any one of Embodiments 116-127, further comprising contacting the substrate with the bicarbonate component, the PBS, or both

Embodiment 129 provides the method of Embodiment 128, wherein contacting the substrate with the bicarbonate component comprises releasing at least one of the substrate and the bicarbonate component, the PBS, or both from a sealed chamber.

Embodiment 130 provides the method of any one of Embodiments 116-129, wherein contacting comprises incubating the analyte and the substrate.

Embodiment 131 provides the method of Embodiment 130, further comprising drying the substrate following incubation but before exposing the substrate to light.

1. A substrate comprising:
a metallic layer; and
a boronic acid component, a nucleic acid aptamer, or both at least partially coating the metallic layer.

2. The substrate of claim 1, wherein the metallic layer comprises a semi-conductive metal.

3. The substrate of claim 1, wherein the metallic layer comprises Ag₂O, elemental silver, elemental gold, elemental copper, elemental platinum, mixtures thereof, alloys thereof, or combinations thereof.

4. The substrate of claim 1, further comprising a polymeric layer attached to the metallic layer.

5. The substrate of claim 1, wherein the boronic acid component is an alkyl boronic acid, an alkenyl boronic acid, a heteroaryl boronic acid, a salt thereof, or a mixture thereof.

6. The substrate of claim 5, wherein the aryl boronic acid is an unsubstituted aryl boronic acid, a monosubstituted aryl boronic acid, a disubstituted aryl bornonic acid, a trisubstituted aryl boronic acid, a tetrasubstitued aryl boronic acid, a pentasubstituted aryl boronic acid, a salt thereof, or a mixture thereof.

7. The substrate of claim 1, wherein the nucleic acid aptamer comprises a deoxyribonucleic acid, a ribonucleic acid, or a mixture thereof.

8. The substrate of claim 1, further comprising an aqueous solution comprising a bicarbonate component, a PBS buffer, or both.

9. The substrate of claim 1, wherein the substrate further comprises a biological component contacting the boronic acid component, the nucleic acid aptamer, or both.

10. The substrate of claim 9, wherein the biological component comprises bacteria.

11. The substrate of claim 10, wherein the bacteria comprises a gram-positive bacteria, a gram-negative bacteria, or a mixture thereof.

12. The substrate of claim 10, wherein the bacteria comprises Clostridium botulinum, Listeria monocytogenes, Acetic acid bacteria, Acidaminococcus, Acinetobacter baunmannii, Agrobacterium tumefaciens, Akkermansia muciniphila, Anaerobiospirillumn, Anaerolinea thermolimosa, Anaerolinea thermophila, Arcobacter, Arcobacter skirrowii, Armatinonas rosea, Azotobacter salinestris, Bacteroides, Bacteroides fragilis, Bacteroides ureolyticus, Bacteroidetes, Bartonella japonica, Bartonella koehlerae, Bartonella taylorii, Bdellovibrio, Brachyspira, Bradyrhizobiumn japonicumn, Caldilinea aerophile, Cardiobacterium hominis, Chaperone-Usher fimbriae, Christensenella, Chthonomonas calidirosea, Coxiella burnetiid, Cyanobacteria, Cytophaga, Dehalogeninonas lykanthroporepellens, Desulfurobacterium atlanticum, Devosia pacifica, Devosia psychrophila, Devosia soli, Devosia subaequoris, Devosia submarina, Devosia yakushimensis, Dialister, Dictvoglomus thermophilum, Enterobacter, Enterobacter cloacae, Enterobacter cowanii, Enterobacteriaceae, Enterobacteriales, Escherichia, Escherichia coli, Escherichia fergusonii, Escherichia hernumannii, Finbriimonas ginsengisoli, Flavobacteriumn, Flavobacteriumn akiainvivens, Francisella novicida, Fusobacterium necrophonim, Fusobacterium nucleatum, Fusobacteriumn polymorphum, Haemophilus felis, Haemophilus haemolyticus, Haemophilus influenzae, Haemophilus pittmaniae, Helicobacter, Kingella kingae, Klebsiella pneumoniae, Kluyvera ascorbate, Kluyvera cryocrescens, Legionella, Legionella clemsonensis, Legionella pneumophila, Leptonema illini, Leptotrichia buccalis, Levilinea saccharolytica, Luteimonas aquatic, Luteimonas composti, Luteimonas lutimaris, Luteimonas marina, Luteimonas mephitis, Luteimonas vadose, Megamonas, Megasphaera, Meiothermus, Meiothermus timidus, Methylobacterium fujisawoense, Morax-Axenfeld diplobacilli, Moraxella, Moraxella bovis, Moraxella osloensis, Morganella morganii, Mycoplasma spumans, Neisseria cinereal, Neisseria gonorrhoeae, Neisseria meningitidis, Neisseria polysaccharea, Neisseria sicca, Nitrosomonas eutropha, Nitrosomonas halophila, Nonpathogenic organisms, OMPdb, Pectinalus, Pedobacter heparinus, Pelosinus, Propionispora, Proteobacteria, Proteus mirabilis, Proteus penneri, Pseudomonas, Pseudomonas aeruginosa, Pseudomonas luteola, Pseudoxanthomonas broegbernensis, Pseudoxanthomonas japonensis, Rickettsia rickettsia, Salinibacter ruber, Salmonella, Salmonella bongori, Salmonella enterica, Samsonia, Selenomonadales, Serratia marcescens, Shigella, Shimwellia, Solobacterium moorei, Sorangium cellulosum, Sphaerotilus natans, Sphingomonas gei, Spirochaeta, Spirochaetaceae, Sporomusa, Stenotrophomonas, Stenotrophomonas nitritireducens, Thermotoga neapolitana, Thorselliaceae, Trimeric autoiransporter adhesion, Vampirococcus, Verminephrobacter, Vibrio adaptatus, Vibrio azasii, Vibrio campbellii, Vibrio cholerae, Victivallis vadensis, Vitreoscilla, Wolbachia, Yersiniaceae, Zymophilus, strains thereof, or combinations thereof.

13. A method of making a substrate, the method comprising coating a metallic layer with a boronic acid component, a nucleic acid aptamer, or both.

14. A method of using a substrate, the method comprising:
contacting the substrate with an analyte, the substrate comprising:
a metallic layer; and
a boronic acid component, a nucleic acid aptamer, or both at least partially coating the metallic layer;
exposing the substrate to light;
capturing an image of the substrate; and
visually confirming the presence or absence of bacteria.

15. The method of claim 14, further comprising magnifying the substrate using a magnifying lens.

16. The method of claim 14, wherein the analyte comprises animal tissue, plant tissue, animal extract, plant extract, or a mixture thereof.

17. The method of claim 14, wherein the analyte comprises bacteria.

18. The method of claim 17, wherein the bacteria comprises a predetermined bacteria.

19. The method of claim 18, wherein the aptamer bonds with a surface protein of the predetermined bacteria.

20. The method of claim 14, wherein the nucleic acid comprises the sequence according to SEQ ID NO: 1 or SEQ ID NO: 2.


SEQUENCE LISTING (2 sequences)

SEQ ID NO: 1 - DNA, 66 bp, Artificial Sequence
  A synthetic DNA aptamer
aaaaaactcctctgactgtaaccacgcacaaaggctcgcgcatggtgtgtacgttcttacagaggt

SEQ ID NO: 2 - DNA, 57 bp, Artificial Sequence
  A synthetic DNA aptamer
ttttttttttatccatggggcggagatgagggggaggagggcgggtacccggttgat